(19) United States
(12) Patent Application Publication
(10) Pub. No.: US 2020/0230170 A1
(43) Pub. Date: Jul. 23, 2020

(54) HYDROGEN-CONTAINING COMPOSITION

(71) Applicants: Jason Madison MILLER, San Clemente, CA (US); MiZ Company Limited, Kamakura-shi, Kanagawa (JP)

(72) Inventors: Yasushi SATOH, Tokorozawa-shi, Saitama (JP); Shinichi HIRANO, Kamakura-shi, Kanagawa (JP); Bunpei SATO, Kamakura-shi, Kanagawa (JP); Ryosuke KUROKAWA, Kamakura-shi, Kanagawa (JP); Jason Madison MILLER, San Clemente, CA (US)

(21) Appl. No.: 16/644,432

(22) PCT Filed: Sep. 14, 2018
(86) PCT No.: PCT/US2018/051011

§ 371 Date: Mar. 4, 2020

(30) Foreign Application Priority Data
Sep. 14, 2017 (JP) 2017-176425

(51) Int. Cl. A61K 33/00; A61K 9/00

(52) U.S. Cl. CPC A61K 33/00; A61K 9/007

(57) ABSTRACT

An object of the present invention is to provide means for treating post-traumatic stress disorder or a blast-induced traumatic brain injury-protective agent. The present invention provides a pharmaceutical composition containing hydrogen as an active ingredient, used for treating post-traumatic stress disorder or protecting against blast-induced traumatic brain injury.

### TECHNICAL FIELD

The present invention relates to a composition for treating or preventing post-traumatic stress disorder (PTSD). The present invention further relates to a method for treating or preventing PTSD, in particular, the present invention relates to a method for treating or preventing PTSD, the method including administering hydrogen gas to a subject.

Furthermore, the present invention relates to a traumatic brain injury-protective agent to ameliorate blast-induced traumatic brain injury caused in sites with military conflicts or terrorism.

### BACKGROUND ART

In recent years, hydrogen gas (molecular hydrogen, H₂) has been introduced as a medical gas in the field of clinical medicine (Non Patent Literature 1). Gas containing hydrogen molecules is known to be applicable as an antioxidant effective for the treatment of ischemia-reperfusion injury and cerebral infarction (Patent Literature 1). When hydrogen gas is inhaled from the lung, hydrogen systemically distributes through diffusion and the blood to prevent diseases associated with reactive oxygen and reductively eliminate highly-oxidative free radicals causing cell disorder, which is the cause for the effectiveness of hydrogen gas for ischemia-reperfusion injury and cerebral infarction. The effect of hydrogen gas provided to the clinical medicine is inferred to be due to the antioxidative action, anti-apoptotic action, and anti-inflammatory action of hydrogen gas (Patent Literature 1).

Post-traumatic stress disorder (PTSD) is a stress disorder induced by being greatly traumatized by a life-threatening event (e.g., wars, natural disasters, accidents, crimes, abuse) to cause intense distress or impaired functioning. If people have directly or indirectly experienced a traumatic event, thereafter various symptoms (intrusion symptoms, avoidance symptoms, negative effects on thinking and mood, and changes in alertness and reactions) have been present for I month or longer and symptoms cause significant distress or significantly impair functioning, then they are diagnosed with PTSD. It is inferred that PTSD affects almost 9% of people sometime during their life, including childhood.

In treating PTSD, psychotherapy (e.g., prolonged exposure therapy) is primarily provided by a specialist, and an SSRI anti-depressant or the like is prescribed to mitigate symptoms.

Blast injury has become a great threat in recent battle areas and areas suffering from terrorism because of the use of improvised explosive devices in military conflicts or terrorism. The blast-induced mild traumatic brain injury (bmTBI) is known to manifest itself as psychological effects.

It has reported that excessive reactive oxygen species (ROS) are generated in rodent models of bmTBI. It has been recently revealed that a hydrogen molecule, a reductant, selectively reacts with hydroxyl radical (—OH) and peroxynitrite (ONOO⁻), which are each highly reactive ROS, and eliminate them through reduction. In addition, it has been demonstrated that because a hydrogen molecule is the smallest molecule without causing any side effects and is electrically neutral, the hydrogen molecule can reach target organs easily, diffuse across cell membranes rapidly into cells, and penetrate the blood-brain barrier for the protection of neurons.

### CITATION LIST

### Patent Literature

[Patent Literature 1] WO2007/021034

### Non Patent Literature

[Non Patent Literature 1] Ohta S., et al., Methods Enzymol. 2015; 555: 289-317.

### SUMMARY OF INVENTION

### Technical Problem

PTSD is a disease which is caused by wars, natural disasters including earthquake disasters, fires, accidents, violence, and crimes and can develop in any individual of any age. However, the treatment method is inadequate, and safe and effective means for treatment of PTSD is strongly demanded.

Since the conflicts in Iraq and Afghanistan, bmTBI has received attention. The risk of bmTBI is increasingly recognized not only in military conflicts but also in terrorism and disasters involving civilians. In particular, the significant psychophysiological impact of bmTBI has become serious among US veterans. However, the mechanism of the psychophysiological impact of blast waves is still unknown, and an effective therapy has been still unestablished, and hence the development of a novel therapy has been desired.

An object of the present invention to solve the problem is to provide a traumatic brain injury-protective agent for ameliorating the psychophysiological impact of bmTBI.

### Solution to Problem

The present inventors have studied to solve the problem, and found that inhalation of hydrogen gas provides ameliorating effect on symptoms in PTSD patients, and completed the following inventions.

[1] A composition containing hydrogen as an active ingredient, used for treating post-traumatic stress disorder.

[2] The composition according to [1], used for treating moderate to severe post-traumatic stress disorder.

[3] The composition according to [1] or [2], used for treating post-traumatic stress disorder with chronic pain.

[4] The composition according to any of [1] to [3], in the form of a hydrogen-containing gas or liquid.

[5] The composition according to any of [1] to [4], as a hydrogen-containing gas to be inhaled by a subject in use.

[6] The composition according to any of [1] to [5], as a hydrogen-containing gas used for pulmonary administration.

[7] The pharmaceutical composition according to any of [1] to [6], in the form of a hydrogen gas-containing gas with a concentration of 4 vol % or more, wherein the hydrogen gas is inhaled.

[8] The pharmaceutical composition according to any one of [1] to [7], in the form of a hydrogen gas-containing gas with a concentration of 6 volt or more, wherein the hydrogen gas is inhaled.

[9] The pharmaceutical composition according to any of [1] to [8], wherein hydrogen gas is inhaled for 30 minutes or longer per day in use.

[10] The pharmaceutical composition according to any of [1] to [9], wherein hydrogen gas is inhaled for 60 minutes or longer per day in use.

[11] The pharmaceutical composition according to any of [1] to [10], wherein inhalation of hydrogen gas is performed over 1 week or longer.

[12] The composition according to any of [1] to [11], for use as a pharmaceutical composition.

[13] A blast-induced traumatic brain injury-protective agent containing a hydrogen-containing gas or liquid.

[14] The traumatic brain injury-protective agent according to [13], wherein the traumatic brain injury-protective agent exhibits ameliorating effect on social behavior impairments and depression-like behaviors.

### BRIEF DESCRIPTION OF DRAWINGS

FIG. 1 is a graph showing the amelioration of symptoms of PTSD after initiation of hydrogen inhalation.

FIG. 2 is a graph showing the amelioration of symptoms of PTSD after initiation of hydrogen inhalation.

FIG. 3 is a schematic diagram of a blast generator used to examine the effect of the present invention.

FIG. 4 is a time schedule for inhalation of hydrogen gas and measurement days in examination of the effect of the present invention.

FIG. 5A is a measurement record of typical pressure-time characteristics of blast (driving pressure: 0.8 MPa) generated by the blast generator, and FIG. 5B is a measurement record of typical shock wave pressure-time characteristics of blast (driving pressure: 2.0 MPa) generated by the blast generator.

FIG. 6 is a photograph of a dissected mouse brain exposed to low-level blast.

FIG. 7A is a histochemical staining image with dihydroethidium (Sham group), FIG. 7B is a histochemical staining image with dihydroethidium (bmTBI group), and FIG. 7C is a histochemical staining image with dihydroethidium (bMTBI+H₂ group).

FIG. 8A is a graph showing the results of an open field test, and FIG. 8B is a graph showing the results of an accelerating rotarod test.

FIG. 9 is a graph showing the results of an elevated plus-maze test.

FIG. 10 is a graph showing the results of a Y-maze test.

FIG. 11A is a graph showing the results of a sociability test, FIG. 11B is a graph showing the results of an olfactory test, and FIG. 11C is a graph showing the results of a novelty test.

FIG. 12A is a graph showing the results of a tail suspension test, and FIG. 12B is a graph showing the results of a forced swim test.

FIG. 13 is a graph showing the amelioration of symptoms of PTSD after initiation of hydrogen inhalation.

FIG. 14 is a table showing the details of scores in cases in Example 1.

FIG. 15 is a table showing the details of scores in cases in Example 2.

FIG. 16 is a table showing the details of scores in cases in Example 3.

### ADVANTAGEOUS EFFECTS OF INVENTION

The present invention provides an approach to treating PTSD, and an approach to mitigating symptoms of PTSD. The approaches provided by the present invention cause fewer problems such as side effects, and are each excellent as an approach used for a long period in patients regularly using analgesics or the like.

In addition, the present invention provides an approach for treating bmTBI, and an approach for mitigating symptoms of bmTBI. The approaches provided by the present invention cause fewer problems such as side effects, and are each excellent as an approach used for a long period in patients regularly using analgesics or the like.

### DESCRIPTION OF EMBODIMENTS

The present invention is used for treating PTSD. PTSD involves symptoms such as re-experience (flashback), avoidance behavior, and hypervigilance (including difficulty falling asleep, irritability, and concentration difficulty) as basic symptoms, and characterized in that the symptoms last for 1 month or longer. PTSD develops in most cases within 6 months after getting a serious shock as a stress factor, and in some cases develops 6 months or later, as “delayed PTSD”. Treatment with the composition according to the present invention may be initiated at any time after a subject is confirmed to present with a symptom of PTSD.

The composition according to the present invention can be applied to patients with moderate or severe PTSD, for example, patients with a symptom lasting 6 months or longer from the onset, specifically, patients with a symptom lasting 1 year or longer from the onset, more specifically, patients with a symptom lasting 2 years or longer from the onset. The seriousness of PTSD can be determined, for example, by using the CAPS (Clinician Administered PTSD Scale) or another scale as an index. An applicable version of the CAPS is that described in “Diagnostic and Statistical Manual of Mental Disorders (DSM)” published by the American Psychiatric Association. The composition according to the present invention can be applied to patients with chronic PTSD.

The composition according to the present invention can be applied not only to patients with PTSD but also to those affected by another disease such as depressive disorder, substance-related disorder, anxiety, and bipolar disorder, and can be used in combination with a therapeutic agent for another disease.

The traumatic brain injury-protective agent according to an embodiment of the present invention consists of a hydrogen-containing gas or liquid, and can be used for blast-induced traumatic brain injury, though the usage is not limited thereto.

It is desirable for the hydrogen-containing gas to contain air or an inhalation anesthetic agent mixed therein and have a hydrogen gas concentration of 10% or less, which is lower than the lower limit concentration for explosion. Hydrogen gas concentrations higher than the lower limit concentration for explosion are not preferred because of the risk of explosion due to static electricity or fire. The hydrogen-containing liquid can be prepared by dissolving hydrogen molecules in water or a liquid for injection such as physiological saline. To take the hydrogen-containing liquid, the hydrogen-containing liquid can be drunken as hydrogen water, or injected as an intravenous drip of an infusion.

In the usage of the traumatic brain injury inhibitor according to the present invention, the hydrogen-containing gas or liquid is inhaled through respiration, orally taken by drinking, or injected as an intravenous drip of an injection solution. Diseases targeted by the present invention are not limited to blast-induced traumatic brain injury, and include social behavior impairments and depression-like behaviors induced by any cause other than blast. Specifically, depression-like behaviors induced by post-traumatic stress disorder (PTSD) are also included in examples of the targeted diseases. The time for inhalation of the hydrogen-containing gas can be, for example, 30 minutes to 8 hours, though the time depends on symptoms in a patient. The frequency of intake can be, for example, once to four times per day for drinking of hydrogen water, and once to three times per day for an intravenous drip.

In an aspect, the composition according to the present invention can be used as a pharmaceutical composition. The method according to the present invention can be performed through inhalation of a composition which can be prepared by a hydrogen generator allowing installation at home. Hence, the composition according to the present invention can be used, in another aspect, in methods not corresponding to medical practice or methods not corresponding to therapeutic practice.

The composition or traumatic brain injury inhibitor according to the present invention contains hydrogen gas as an active ingredient. In a mode of the present invention, the hydrogen gas is used as a form of a hydrogen gas-containing gas.

The hydrogen gas-containing gas can be, for example, air containing hydrogen gas, or a mixed gas of hydrogen gas and oxygen gas. The concentration of the hydrogen gas in the hydrogen gas-containing gas is, for example, 18.3 vol % or lower, which is lower than the lower limit concentration for detonation of hydrogen gas, for example, 0.5 to 18.3 vol %, specifically 1 to 10 vol %, more specifically, 2 to 8 vol%, even more specifically 3 to 6 vol %, even more specifically 4 to 6 vol %, and even more specifically 4 to 5 vol % but not especially limited thereto. The concentration of the hydrogen gas in the hydrogen gas-containing gas is preferably 1 to 10 vol %, 2 to 9 vol %, 3 to 8 vol %, and more preferably 3 to 5 vol %.

If the gas other than the hydrogen gas is air, the concentration of the air is, for example, in the range of 81.7 to 99.5 vol %. If the gas other than the hydrogen gas is oxygen gas, the concentration of the oxygen gas is, for example, in the range of 21 to 99.5 vol %. The hydrogen gas-containing gas may contain two or more gasses in addition to hydrogen, and examples thereof include gases such as air, oxygen gas, nitrogen gas, and carbon dioxide gas. Since hydrogen gas is flammable and explosive, it is preferred from the viewpoint of safety to use hydrogen gas with a concentration below the explosion limit of hydrogen gas.

The flow rate of the hydrogen gas composition diluted to a safe concentration for inhalation can be, for example, 1 to 10 L/min, 1 to 6 L/min, 1 to 4 L/min, specifically 2 to 4 L/min, and can be 6 to 8 L/min for patients with hyperpnoea. In a mode of the present invention, inhalation of the hydrogen gas composition can be performed with a flow rate of 70 ml/min or higher, 80 ml/min or higher, 140 ml/min or higher, or 280 ml/min or higher in terms of the amount of hydrogen contained in the composition.

The hydrogen gas-containing gas is blended so as to have a given hydrogen gas concentration, and then can be stored in a pressure-resistant container (e.g., an aluminum can, a plastic bottle) by filling. Alternatively, the hydrogen gas-containing gas may be prepared in situ by using a known hydrogen gas supply apparatus and used for inhalation.

The hydrogen gas supply apparatus enables mixing of hydrogen gas generated from the reaction between a hydrogen-generating agent (e.g., metal aluminum) and water and dilution gas (e.g., air, oxygen) with a given ratio (Japanese Patent No. 5228142), or enables mixing of hydrogen gas generated through electrolysis of water and dilution gas (Japanese Patent No. 5502973, and Japanese Patent No. 5900688). Thereby, a hydrogen gas-containing gas with a hydrogen concentration in the range of 0.5 to 18.5 vol % can be prepared.

In the present invention, hydrogen gas is administered to a subject through inhalation. The hydrogen gas administered is expected to be absorbed from the mucosa at the respiratory tract or lung in the way to the lung after administration. The hydrogen gas incorporated from the lung and so forth can be systemically delivered to each tissue not only via the blood, but also through diffusion from the lung. The composition according to the present invention may be used for pulmonary administration.

In inhalation of hydrogen gas, a mask-type device to cover the mouth and nose, a nasal cannula or the like can be used.

Hydrogen gas incorporated in a living body through inhalation distributes much in the brain, lung, and muscle, and the amount of intratissue hydrogen (Area under the curve: AUC) is larger than those in other methods of administration such as oral administration, intraperitoneal administration, and intravenous administration. Here, comparison is made between drinking of hydrogen water by a human and inhalation of hydrogen gas by a human. In drinking of hydrogen water, most of the hydrogen molecules reach tissues and organs in the abdominal portion through diffusion from the stomach and intestinal tract, and a part of the hydrogen molecules is absorbed from the intestinal wall and distributed in tissues and organs in the whole body through the blood circulation. In inhalation of hydrogen gas, on the other hand, the hydrogen molecules are transferred through the following pathways: [1] the hydrogen molecules are mixed with inhaled air and transferred to the lung tissue, and distributed to the surrounding tissues through diffusion; [2] the hydrogen molecules are dissolved in the blood through pulmonary gas exchange and systemically transferred; and [3] the hydrogen molecules are directly transferred to the brain tissues via the nasal mucosa without passing through the blood-brain barrier (BBB).

The gas with the above hydrogen concentration may be administered once or a plurality of times (e.g., twice or three times) per day. The period of administration can be set to, for example, 1 week or longer, 2 weeks or longer, 4 weeks or longer, 2 months or longer, 3 months or longer, 6 months or longer, 1 year or longer, 2 years or longer, or 3 years or longer. The time of administration in one administration can be set to, for example, 5 minutes or longer, 10 minutes or longer, 15 minutes or longer, 20 minutes or longer, 30 minutes or longer, 40 minutes or longer, 1 hour or longer, 2 hours or longer, 3 hours or longer, or 4 hours or longer. The time of administration may be continuous or divided into a plurality of portions. The period of administration and time of administration may be appropriately set in view of the condition of a subject. Inhalation of hydrogen gas is suitable for long-term treatment because of fewer burdens on subject's health and few adverse events reported. In a mode of the present invention, treatment with the composition according to the present invention can be continued until symptoms of PTSD are mitigated or disappear.

Alternatively, a subject can take hydrogen gas successfully through staying in a space filled with a hydrogen gas-containing gas for a given period of time. The pressure in the space may be the standard atmospheric pressure (approximately 1.013 atm), or a raised pressure in the range of higher than the standard atmospheric pressure and 7 atm or lower, for example, 1.02 atm to 7.0 atm, specifically 1.02 atm to 5.0 atm, more specifically 1.02 atm to 4.0 atm, and even more specifically 1.02 atm to 1.35 atm. Intake of hydrogen under a high-pressure environment is preferred in that the body absorption of hydrogen is promoted in a subject. For intake of hydrogen under pressure, a high-pressure capsule designed to have a sufficient strength can be used.

The term “subject” herein encompasses mammals, for example, primates including humans; rodents such as mice and rats; companion animals such as dogs and cats, and animals for viewing such as animals bred in zoos. Humans are preferred subjects in the present invention.

### EXAMPLES

Hereinafter, the present invention will be described in detail by using reference examples and Examples. However, the present invention is never limited to these Examples.

### Example 1

Adult women who had lived for 10 years or longer after the onset of PTSD caused by abuse in early childhood were subjected to inhalation of hydrogen gas with a concentration of 7% by using a hydrogen generator (MHG-2000α, produced by MiZ Company Limited) for at least 1 hour (up to 4 hours) per day over 7 weeks. Each of the subjects had been regularly using an analgesic prescribed by a physician to treat pain, and continued the regular use also during the period of inhalation.

Symptoms of PTSD were quantified through interviews by a physician with the PTSD Checklist for DSM-5 (PCL-5; published by the National Center for PTSD). The contents of the PCL-5 are shown in the following table.

TABLE 1
Instructions: Below is a list of problems that people sometimes have in response to a very stressful
experience. Please read each problem carefully and then circle one of the numbers to the right to
indicate how much you have been bothered by that problem in the past month.

| | | Not at | A little | | Quite | |
|---|---|---|---|---|---|---|
| In the past month, how much were you bothered by: | | all | bit | Moderately | a bit | Extremely |
| 1. | Repeated, disturbing, and unwanted memories of the | 0 | 1 | 2 | 3 | 4 |
| | stressful experience? | | | | | |
| 2. | Repeated, disturbing dreams of the stressful experience? | 0 | 1 | 2 | 3 | 4 |
| 3. | Suddenly feeling or acting as it the stressful experience were | | | | | |
| | actually happening again (as if you were actually back there | 0 | 1 | 2 | 3 | 4 |
| | reliving it)? | | | | | |
| 4. | Feeling very upset when something reminded you of the | 0 | 1 | 2 | 3 | 4 |
| | stressful experience? | | | | | |
| 5. | Having strong physical reactions when something reminded | 0 | 1 | 2 | 3 | 4 |
| | you of the stressful experience (for example, heart | | | | | |
| | pounding, trouble breathing, sweating)? | | | | | |
| 6. | Avoiding memories, thoughts, or feelings related to the | 0 | 1 | 2 | 3 | 4 |
| | stressful experience? | | | | | |
| 7. | Avoiding external reminders of the stressful experience (for | 0 | 1 | 2 | 3 | 4 |
| | example, people, places, conversations, activities, objects, or | | | | | |
| | situations)? | | | | | |
| 8. | Trouble remembering important parts of the stressful | 0 | 1 | 2 | 3 | 4 |
| | experience? | | | | | |
| 9. | Having strong negative beliefs about yourself, other people, | 0 | 1 | 2 | 3 | 4 |
| | or the world (for example, having thoughts such as: I am | | | | | |
| | bad, there is something seriously wrong with me, | | | | | |
| | no one can be trusted, the world is completely dangerous)? | | | | | |
| 10. | Blaming yourself or someone else for the stressful | 0 | 1 | 2 | 3 | 4 |
| | experience or what happened after it? | | | | | |
| 11. | Having strong negative feelings such as fear, horror, anger, | 0 | 1 | 2 | 3 | 4 |
| | guilt, or shame? | | | | | |
| 12. | Loss of interest in activities that you used to enjoy? | 0 | 1 | 2 | 3 | 4 |
| 13. | Feeling distant or cut off from other people? | 0 | 1 | 2 | 3 | 4 |
| 14. | Trouble experiencing positive feelings (for example, being | 0 | 1 | 2 | 3 | 4 |
| | unable to feel happiness or have loving feelings for people | | | | | |
| | close to you)? | | | | | |
| 15. | Irritable behavior, angry outbursts, or acting aggressively? | 0 | 1 | 2 | 3 | 4 |
| 16. | Taking too many risks or doing things that could cause you | 0 | 1 | 2 | 3 | 4 |
| | harm? | | | | | |
| 17. | Being “superalert” or watchful or on guard? | 0 | 1 | 2 | 3 | 4 |
| 18. | Feeling jumpy or easily startled? | 0 | 1 | 2 | 3 | 4 |
| 19. | Having difficulty concentrating? | 0 | 1 | 2 | 3 | 4 |
| 20. | Trouble falling or staying asleep? | 0 | 1 | 2 | 3 | 4 |

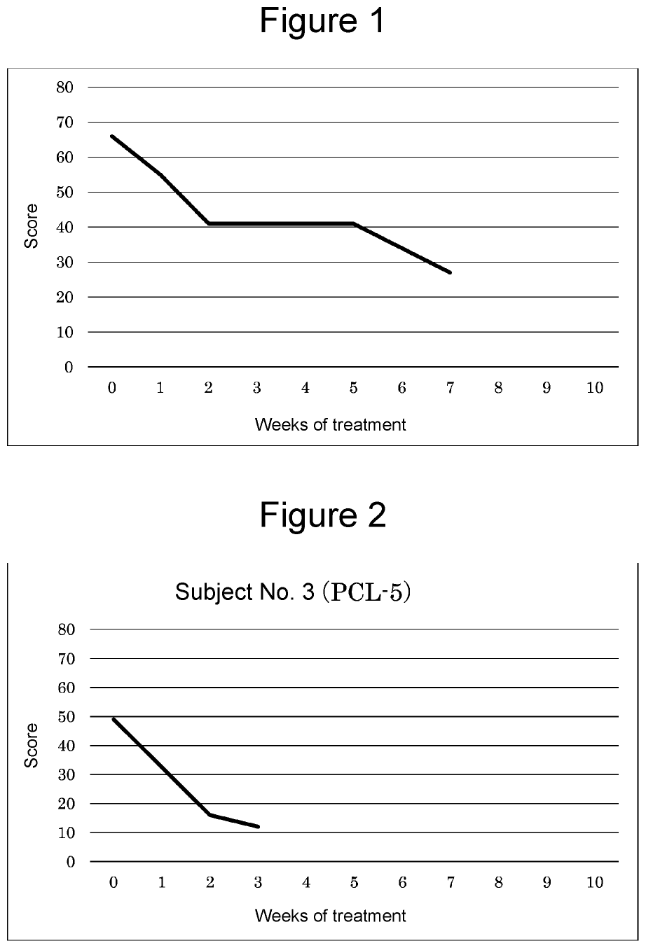

Table 2 shows average inhalation times and total scores by the PCL-5 at respective weeks, and FIG. 1 shows the graph. It was found that symptoms of PTSD tended to ameliorate after the initiation of inhalation.

TABLE 2

| | Average | |
|---|---|---|
| | inhalation | |
| Treatment period | time/day | Total score |
| Before inhalation | 0 | 66 |
| Week 1 | 2.2 H | 55 |
| Week 2 | 3.8 H | 41 |
| Week 3 | 2.8 H | — |
| Week 4 | 2.8 H | 41 |
| Week 5 | 1.1 H | — |
| Week 6 | 1.1 H | — |
| Week 7 | 1.1 H | 27 |

### Example 2

Adult women who had experienced the onset of PTSD due to an intense psychological shock received in military operations were subjected to inhalation of hydrogen gas with a concentration of 7% by using a hydrogen generator (MHG-2000α, produced by MiZ Company Limited) for at least 2 hours (up to 4 hours) per day over 3 weeks. Each of the subjects had been regularly using an analgesic prescribed by a physician to treat pain, and continued the regular use also during the period of inhalation.

Symptoms of PTSD were quantified through interviews by a physician. Symptoms after the initiation of inhalation are shown in FIG. 2 and Table 3. It was found that symptoms of PTSD tended to ameliorate after the initiation of inhalation.

TABLE 3

| | Average | |
|---|---|---|
| | inhalation | |
| Treatment period | time/day | Total score |
| Before inhalation | 0 | 49 |
| Week 1 | 4 H | — |
| Week 2 | 4 H | 16 |
| Week 3 | 2 H | 12 |

### Example 3

Adult women who had experienced the onset of PTSD due to an intense psychological shock received through physical abuse in early childhood and later stages were subjected to inhalation of hydrogen gas with a concentration of 7% by using a hydrogen generator (MHG-2000α, produced by MiZ Company Limited) for 1 to 3 hours per day over 3 weeks. Each of the subjects had been regularly using an analgesic prescribed by a physician to treat pain, and continued the regular use also during the period of inhalation.

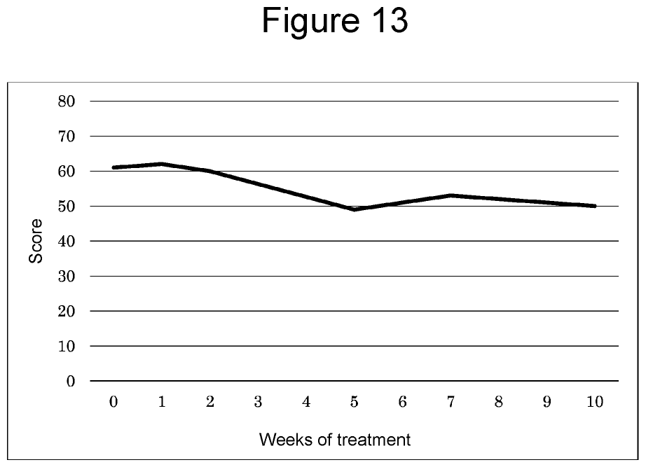

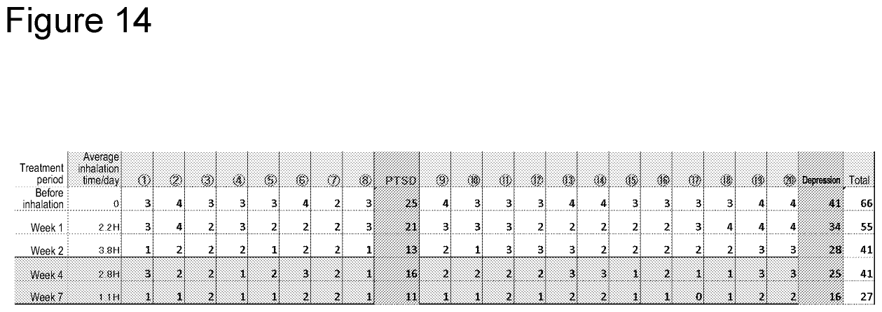

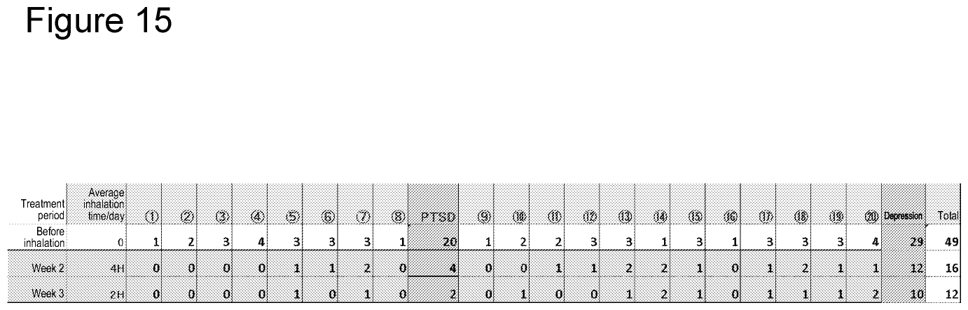

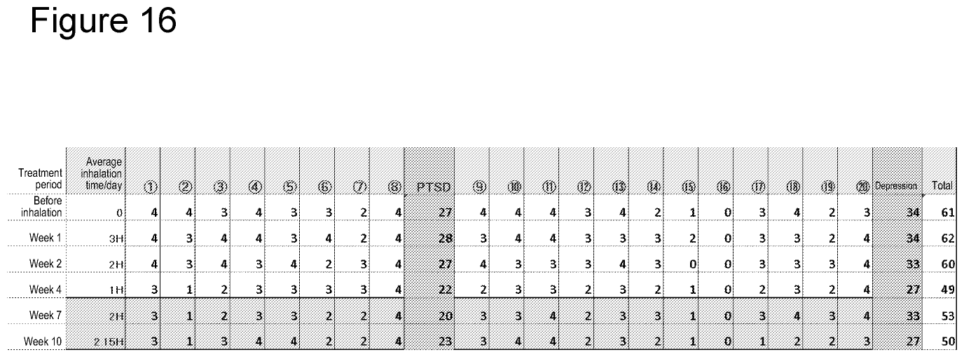

Symptoms of PTSD were quantified through interviews by a physician. Symptoms after the initiation of inhalation are shown in FIG. 13 and Table 4.

TABLE 4

| | Average | |
|---|---|---|
| | inhalation | |
| Treatment period | time/day | Total score |
| Before inhalation | 0 | 61 |
| Week 1 | 3 H | 62 |
| Week 2 | 2 H | 60 |
| Week 4 | 1 H | 49 |
| Week 5 | 2.8 H | — |
| Week 7 | 2 H | 53 |
| Week 10 | 2.15 H | 50 |

### Example 4

### Experimental Animals

All animal experiments relating to the present invention were conducted according to the institutional ethical guidelines for animal experiments of the National Defense Medical College and these animal experiments were approved by the Committee for Animal Research at the National Defense Medical College. Inbred C57BL/6 mice were used in this experiment. Age-matched male littermate mice were used in all experiments. The mice were housed in a breeding room with a 12-h light/dark cycle and a room temperature maintained at 24±1° C. The mice had ad libitum access to water and food.

### Shock Tube Settings

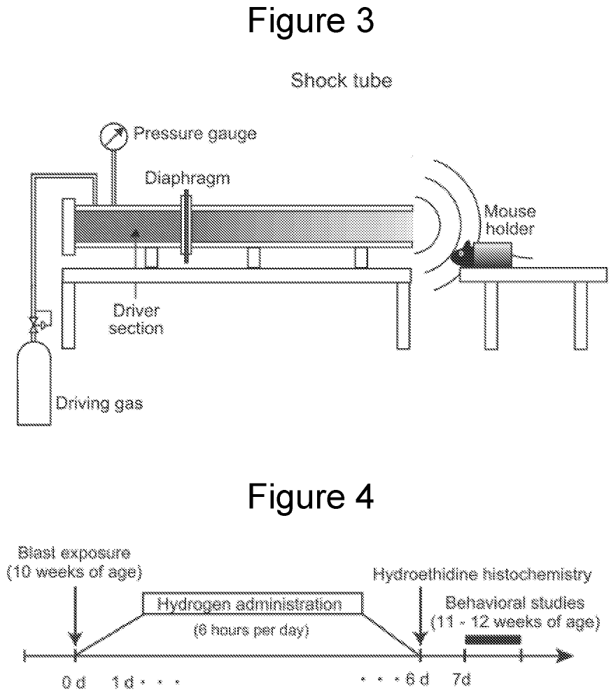

The present inventors designed a compressed gas-driven shock tube with easy adaptability to a laboratory setting. The shock tube included driver and driven sections, which were separated by a polyester diaphragm as shown in FIG. 3. The driver section was filled with compressed nitrogen gas through a gas inlet valve, which was closed once the driver section was filled with the gas. When triggered, a needle raptured the polyester diaphragm, which caused the gasses of the driver section to expand rapidly, forming a blast wave as it traveled along the driven section.

### Shock Wave Exposure

The mice at 10 weeks of age were anesthetized by intraperitoneal administration of mixed anesthetic agents containing medetomidine hydrochloride (0.3 mg/kg, Domitol, Meiji Seika Pharma Co., Ltd.), midazolam (4.0 mg/kg, Dormice, Astellas Pharma Inc.), and butorphanol (5.0 mg/kg, Vetorphale, Meiji Seika Pharma Co., Ltd.). As shown in FIG. 3, each mouse was placed to fix on the mouse holder at a distance of 5 cm from the exit of the shock tube, with the head directed towards the shock tube and the long axis of the body parallel to the shock tube. Following blast exposure, mice were allowed to recover from anesthesia and were subsequently returned to their home cage.

### Shock Wave Recording

The pressure in the shock wave was measured at a resonance frequency of 0.5 MHz via a piezoelectric sensor (PCB 113B26, PCB Piezotoronics, Inc., Depew, N.Y.). The analog output from the piezoelectric sensor was analyzed and recorded by an oscilloscope (DS07104A, Agilent Technologies, Santa Clara, Calif.) with 20-MSa/s sample rate.

### Hydrogen Gas Inhalation

As shown in FIG. 4, mice were allowed to inhale hydrogen gas for 7 days starting immediately after the blast exposure. Mice were placed in a humid chamber for hydrogen gas inhalation for 6 hours per day. Hydrogen gas diluted by air (4%) was supplied to the chamber from a hydrogen gas supply apparatus (MiZ Company Limited) at a feeding rate of 2 L/min. The mice had ad libitum access to water and food in the chamber.

### Evaluation of ROS Formation

To evaluate ROS formation, histochemical staining with hydroethidine (dihydroethidium) was performed. Mice were sacrificed 6 days after the blast exposure and the brains were removed and then immediately cryopreserved. The brains were cut into 10 μm cryosections and placed onto glass slides, washed in double distilled water for 3 minutes, and left to stand in 1 μM dihydroethidium (DHE) solution for 30 minutes in the dark. The oxidation product, ethidium, was generated from DHE by ROS and accumulated within cells. The glass slides were subsequently washed and ethidium-derived red fluorescence was observed by using a fluorescence microscope (Nikon Corporation) with an electron-multiplying (EM) CCD camera (ImagEM, Hamamatsu Photonics K. K.) connected thereto. Samples from four slides obtained from four mice per group were used to experiments.

### Behavioral Analysis

Behavioral analysis was performed 7 days after the blast exposure. All mice used in the behavioral analysis were age-matched male littermates. The behavior of each mouse was monitored and analyzed using a computer-operated video tracking system (SMART, Panlab, Barcelona, Spain), unless otherwise stated. In tests using an apparatus with arms, arm entry was counted when all four legs of a mouse entered each arm. The apparatus was washed after each test. Behavioral studies were performed for mice at 11 to 12 weeks of age.

### Open Field Test

Emotional responses to a novel environment were measured in an open field test. The activity of each mouse was measured as the total distance traveled (m) in 10 minutes.

### Rotarod Test

The rotarod (O'Hara & Co., Ltd.) test is a method to evaluate motor coordination and balance with a rotating rod. This method is used to screen for motor deficits that may influence performance on other behavioral tests. Mice were individually placed on the rod, once they were balanced, the rod was accelerated from 2.5 to 70 r.p.m. over the course of 4 minutes. Mice had to keep their balance on the rotating rod. The accelerated rod eventually forced mice to fall from or roll on the rotarod, and the latency was recorded. Three consecutive experiments were performed at least 30 minutes interval.

### Elevated Plus-Maze Test

An elevated plus-maze test was performed by using a conventional method. Typically, mice prefer a closed environment over an open environment. The evaluation was performed using the percentage (95) of time spent in the open arms as an index of anxiety-like behavior.

### Y-Maze Test

A Y-maze test was performed by using a conventional method. This test serves as an evaluation method of spatial working memory. Mice at 11 weeks of age were used.

### Sociability Test

A sociability test was performed by using a conventional method. Interaction time with another living mouse versus a stuffed mouse was measured in an open field using two cylinder cages allowing olfactory and minimal tactile feelings. The cylinder cages were 10 cm in height, with a bottom diameter of 9 cm and bars spaced 7 mm apart.

### Olfactory Test

An olfactory test was performed by using a conventional method. Briefly, mice were habituated to the flavor of a novel food (blueberry cheese), and, thereafter, subjected to food deprivation for 48 hours. A piece of blueberry cheese was buried under 2 cm of bedding in a clean cage, and the time required to find the buried blueberry cheese was measured for each mouse.

### Novelty Test

A novelty test was performed by using a conventional method. The activity of each mouse was measured as the total duration of interaction with an inanimate novel object (red tube) for 10 minutes.

### Tail Suspension Test

A tail suspension test, an evaluation method of depression-like behavior, was performed. Briefly, a mouse was suspended from the edge of a desk by attaching its tail with adhesive tape. The adhesive tape was placed approximately 5 to 10 mm from the tip of the tail. The suspended mouse was 600 mm away from the floor. The duration of immobility (lack of movement of paws, with the head pointed downward) was measured for the mouse for 6 minutes.

### Forced Swim Test

A forced swim test, known as a behavioral despair test that evaluates depression-like behavior in rodents, was performed by using a conventional method. Briefly, mice were individually placed in a cylinder (25 cm in diameter, 46 cm in depth) filled two-thirds with water (25±1° C.) for 6 minutes. The mice cannot escape from the cylinder, and they cannot put their hind legs on the bottom. Mice swim in the water in search for an escape route. This test was performed 24 hours after the tail suspension test. For each mouse, the time spent swimming versus the time spent floating was measured. The swimming behavior was defined as active horizontal movement more than necessary to merely maintain the head above the water, and was measured as a parameter to assess “hopefulness”. The floating behavior without movement beyond those necessary to maintain balance and keep the nose above the water was measured as a parameter to assess “hopelessness”, a sign of depression-like behavior. After each test, mice were lightly towel-dried and returned their home cage. The water in the cylinder was exchanged for each test.

### Statistical Analysis

Statistical analysis was performed for measurements using statistical analysis software (GraphPad Prism 6.0, GraphPad Software Inc., San Diego, Calif.). Specifically, Student's t-test was used for two-group comparisons; and one-way analysis of variance (One-way ANOVA) or two-way repeated measures ANOVA was performed for multi-group comparisons, and if any statistically significant difference was found in these tests, Bonferroni post-hoc test, a multiple comparison test, was performed to examine the statistically significant difference among the control group, bmTBI group, and bmTBI+H₂ group. Measurement data were presented as mean±standard error, and p<0.05 was considered statistically significant in intergroup comparison.

### Results

### Shock Wave Analysis

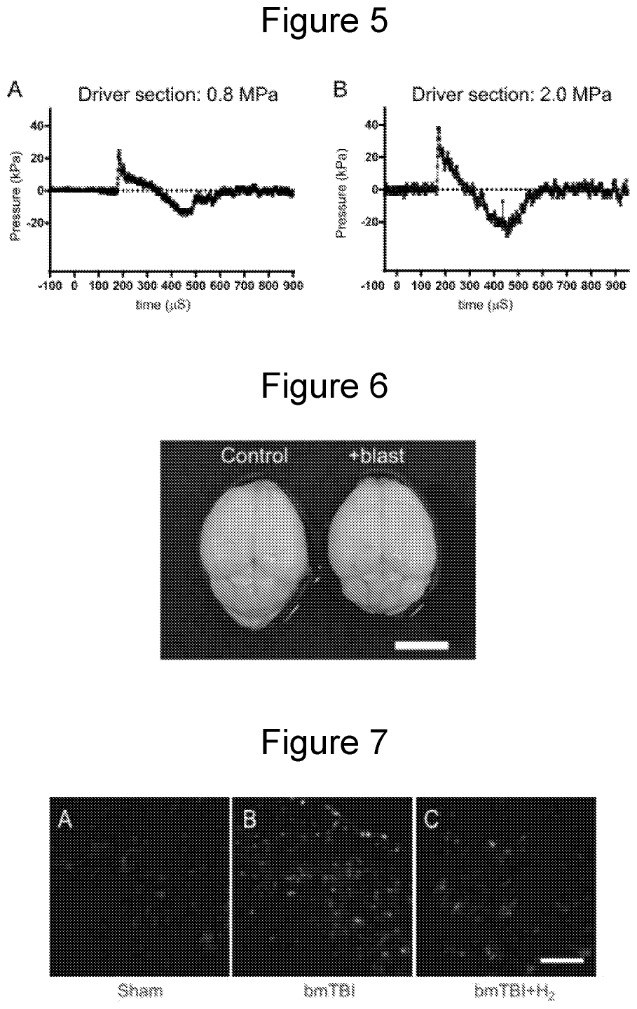

FIG. 5 shows the pressure-time profiles of blast waves driven at 0.8 MPa and 2.0 MPa measured with the pressure sensor placed at the measurement point (mouse head). The peak pressures of the blast waves were 25.0 kPa and 38.0 kPa for the pressures of 0.8 MPa and 2.0 MPa measured at the driver section, respectively (FIG. 5). The waveform was composed of a steep front corresponding to the shock wave, followed by decay of pressure and negative pressure phase (FIG. 5). The present inventors confirmed that an almost linear correlation between the pressure of the driver section and mean peak pressure in the range of 20 to 60 kPa. The generated pressure waves and mean peak wave for each pressure of the driver section were consistent in form and reproducible. The present inventors used a shock wave at 25 kPa generated at 0.8 MPa in the behavioral study in this study.

### Visual Observation of Mouse Brain

Shock of 25.0 kPa did not induce apparent cerebral hemorrhage as shown in FIG. 6; nevertheless, we could not exclude the possibility of brain edema, vasospasm, intracranial hemorrhage, cortical cell loss, and axonopathy.

### Evaluation of ROS Formation

To evaluate ROS formation, histochemical staining with hydroethidine was performed 6 days after blast exposure. FIG. 7 shows that much greater ROS was formed by blast exposure (bmTBI, FIG. 7B) compared with controls (Sham, FIG. 7A). In contrast, ROS was significantly reduced in the brains of mice subjected to inhalation of hydrogen after blast exposure (bmTBI+H₂, FIG. 7C) compared with blast exposure alone (bmTBI, FIG. 7B). These results indicate that exposure to a shock wave induces oxidative stress in the brains and that inhalation of hydrogen gas mitigates this effect.

### Influence on General Behavior

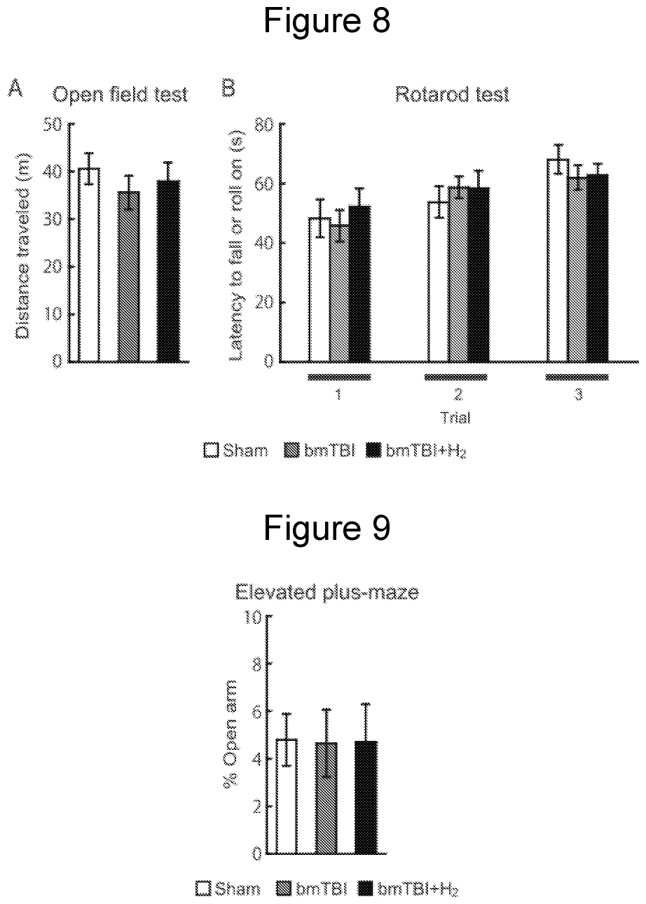

To evaluate the behavioral activity in a novel environment, the open field test was performed. Compared with sham controls (Sham), mice exposed to blast (bmTBI) did not show abnormal activity regardless of inhalation of hydrogen gas, as evaluated by the total distance traveled (FIG. 8A, one-way ANOVA, p>0.05).

Next, to evaluate the gross motor function, mice were subjected to an accelerating rotarod test. None of the mice exposed to blast with inhalation of hydrogen gas (bmTBI+H₂) or without (bmTBI) exhibited deficits in the accelerating rotarod test (FIG. 8B, two-way ANOVA, p>0.05), indicating that the gross motor function was intact in mice exposed to blast regardless of hydrogen inhalation.

### Influence on Anxiety-Related Behavior

To assess whether blast exposure affected anxiety-related behaviors, an elevated plus-maze test was performed. In this test, anxiety-related behavior was assessed by the percentage of time spent in the open arms. As a result, mice in the control (Sham), bmTBI, and bmTBI+H₂ groups did not differ significantly in the percentage of time spent in the open arms (FIG. 9, one-way ANOVA, p>0.05). These results indicate that the anxiety-related behaviors of the mice were not affected by blast exposure regardless of inhalation of hydrogen gas.

### Influence on Spatial Working Memory

Working memory is the ability to hold information temporarily to perform complex cognitive tasks. Mice were subjected to a Y-maze test to evaluate spatial working memory. This test examines whether mice remember the preceding arm position selected. By nature, rodents seek a different arm from that of the preceding choice. However, if working memory is impaired, the number of correct choices should be reduced.

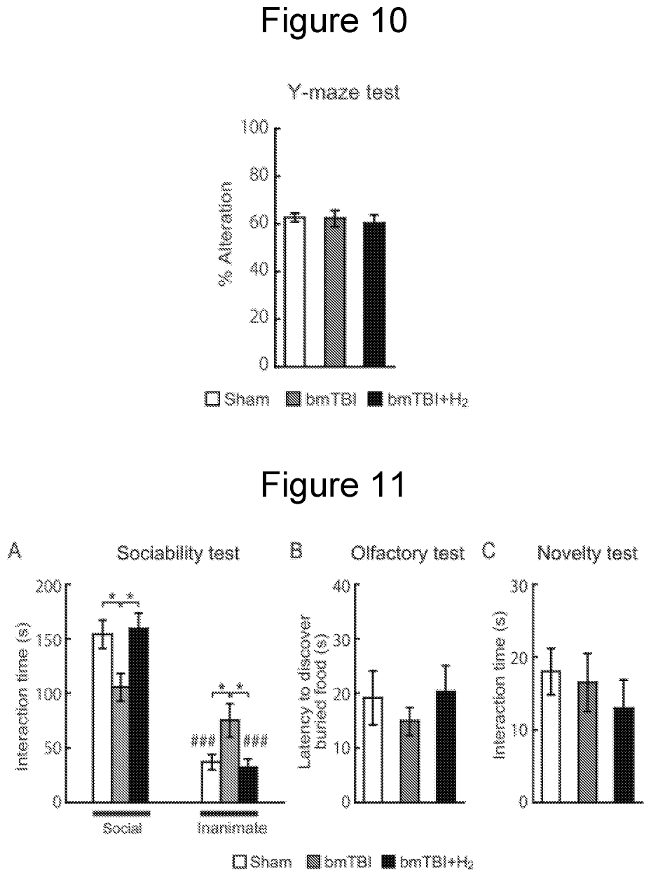

As a result, mice in the control (Sham), bmTBI, and bmTBI+H₂ groups performed this task with 62.6±1.8%, 62.3±3.4%, and 60.4±3.4% correct choices, respectively (FIG. 10, one-way ANOVA, p>0.05). The values for control (Sham), bmTBI, and bmTBI+H₂ groups were well above those expected for random choices (random choice=50%, t-test, p<0.0001 in the above cases).

### Influence on Social Behavior (1)

Mice are a social species and exhibit social interaction behaviors. A sociability test was performed to examine interaction time with a social object (living mouse) versus inanimate object (stuffed mouse). As a result, mice exposed to blast (bmTBI group) exhibited decreased interaction time with a living mouse compared with the control (Sham) group (FIG. 11A). On the other hand, the bmTBI+H₂ group exhibited a similar interaction time with a living mouse to the control group (FIG. 11A). A one-way ANOVA confirmed these differences as statistically significant (F=5.02, p=0.014).

Moreover, mice exposed to blast (bmTBI group) exhibited an increase in the interaction time with a stuffed mouse compared with the control (Sham) group, while the bmTBI+H₂ group exhibited a similar interaction time to the control group (FIG. 11A, one-way ANOVA, F=5.05, p=0.013).

Although mice in the control (Sham) and bmTBI groups spent more interaction time with the living mouse than with the stuffed mouse (FIG. 11A, t-test, both ps<0.001), no significant difference was found between the two interaction times in the bmTBI+H₂ group (FIG. 11A, t-test, p>0.05).

### Influence on Social Behavior (2)

While differences were found in the sociability test, no difference was found in the olfactory test (FIG. 11B, one-way ANOVA, p>0.05) or in the novelty test (FIG. 11C, one-way ANOVA, p>0.05). Therefore, it can be concluded that inhalation of hydrogen gas suppressed the abnormal social behaviors induced by blast.

### Influence on Depression-Like Behavior (1)

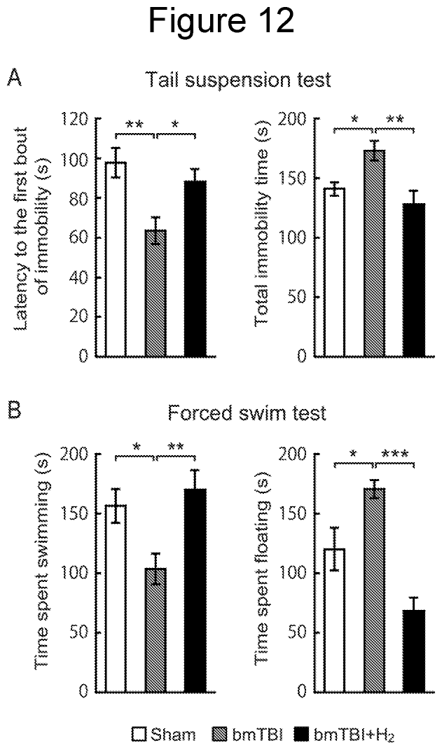

To investigate whether blast exposure induces a depression-like behavior, mice were subjected to the tail suspension test, a widely used method for evaluating depression-like behavior. In this test, the latency to the first bout of immobility and the total immobility time during the 6-minute trial were evaluated. However, mice subjected to inhalation of hydrogen gas after blast exposure (bmTBI+H₂ group) exhibited latencies comparable to the control (Sham) group (FIG. 12A left). A one-way ANOVA confirmed these differences as statistically significant (F=6.42, p=0.0051).

Moreover, mice exposed to blast (bmTBI group) exhibited an increase in the immobility time compared with control mice (Sham), while mice subjected to inhalation of hydrogen gas (bmTBI+H₂ group) exhibited a comparable immobility time to control mice (Sham) (FIG. 12A right, one-way ANOVA, F=6.94, p=0.0036).

### Influence on Depression-Like Behavior (2)

Mice were subjected to the forced swim test, another evaluation method of depression-like behavior. Mice exposed to blast (bmTBI group) exhibited a decrease in the active swimming time compared with control mice (Sham group), while mice subjected to hydrogen gas inhalation (bmTBI+H₂ group) exhibited a similar swimming time to control mice (Sham group) (FIG. 12B left, one-way ANOVA, F=5.95, p=0.059).

Moreover, mice exposed to blast (bmTBI group) exhibited an increase in floating time compared with control mice (Sham group), while mice subjected to inhalation of hydrogen gas (bmTBI+H₂ group) exhibited a similar floating time to control mice (Sham group) (FIG. 12B right, one-way ANOVA, F=11.52, p<0.0001). These results indicate that inhalation of hydrogen gas suppresses depression-like behaviors induced by blast exposure.

### Discussion

In this study, the present inventors demonstrated that inhalation of hydrogen gas markedly suppressed social behavior impairments and depression-like behaviors induced by blast exposure. In addition, this result was supported by the result of immunohistochemical analysis that inhalation of hydrogen gas inhibited oxidative stress due to blast exposure. Collectively, it is likely that hydrogen gas suppresses aberrant behaviors induced by blast exposure through its antioxidant effects.

In the present study, the present inventors used a peak pressure of a shock wave lower than 30 kPa. Although the detailed mechanism of behavioral deficits caused by such a low-level shock wave was not investigated in the study of the present invention, it is suggested in the present invention that oxidative stress plays a key role in the mechanism of behavioral deficits. It is known that exposure of rats to a shock wave at a low-level (10 to 60 kPa) increases the intracranial pressure in a dose-dependent manner to impair the cognitive function. In addition, it is known that exposure of rats to a shock wave as low as 10 kPa induced a reduction in the cognitive function. Although the relevance between the elevated intracranial pressure and oxidative stress is unknown, such a low-level shock wave probably causes behavioral deficits. In contrast, the study by the present inventors showed that exposure to shock waves did not induce impaired working memory in the Y-maze test, but induced social behavior impairment and depression-like behavior. Since the Y-maze test examines short-term spatial memory, which differs from long-term memory in principle, the low-level shock wave probably caused no influence in the Y-maze test.

In the present invention, no examination was made on whether or not inhalation of hydrogen gas would ameliorate the impairments induced by higher peak pressure of a shock wave. Furthermore, the interspecies differences may not allow for direct extrapolation of the present findings to humans. Nevertheless, it is still worth noting from a therapeutic viewpoint of no side effects of hydrogen that inhalation of hydrogen gas after low-level blast exposure significantly mitigated social behavior impairments and depression-like behaviors. Many previous studies have shown that hydrogen can be used as an effective antioxidant for the treatment of various diseases including myocardial ischemia-reperfusion injury, arteriosclerosis, carcinogenesis, neurodegenerative disorders, and hearing disorders. In these studies, no serious side effect of hydrogen was reported at clinical concentrations.

Considering that the ultimate use of hydrogen will be on the battle fields, hydrogen gas cylinders would be very dangerous because of the risk of explosion due to a hit of a shell. However, the present inventors used an electrolysis method in the present study, which can supply hydrogen gas easily and safely, via the electrolysis of water. Furthermore, this electrolyzes is portable. Thus, the present inventors concluded that inhalation of hydrogen gas is a promising therapeutic strategy to mitigate blast-induced traumatic brain injury.

1. A composition comprising hydrogen as an active ingredient, used for treating post-traumatic stress disorder.

2. The composition according to claim 1, used for treating moderate to severe post-traumatic stress disorder.

3. The composition according to claim 1, used for treating post-traumatic stress disorder with chronic pain.

4. The composition according to claim 1, in the form of a hydrogen-containing gas or liquid.

5. The composition according to claim 1, as a hydrogen-containing gas to be inhaled by a subject in use.

6. The composition according to claim 1, as a hydrogen-containing gas used for pulmonary administration.

7. The composition according to claim 1, in the form of a hydrogen gas-containing gas with a concentration of 4 vol % or more, wherein the hydrogen gas is inhaled.

8. The composition according to claim 1, in the form of a hydrogen gas-containing gas with a concentration of 6 vol % or more, wherein the hydrogen gas is inhaled.

9. The composition according to claim 1, wherein hydrogen gas is inhaled for 30 minutes or longer per day in use.

10. The composition according to claim 1, wherein hydrogen gas is inhaled for 60 minutes or longer per day in use.

11. The composition according to claim 1, wherein inhalation of hydrogen gas is performed over 1 week or longer.

12. The composition according to claim 1, for use as a pharmaceutical composition.

13. A blast-induced traumatic brain injury-protective agent comprising a hydrogen-containing gas or liquid.

14. The traumatic brain injury-protective agent according to claim 13, wherein the traumatic brain injury-protective agent exhibits ameliorating effect on social behavior impairments and depression-like behaviors.
